(19) United States
(12) Patent Application Publication
(10) Pub. No.: US 2024/0344090 A1
(43) Pub. Date: Oct. 17, 2024

(54) PROCESS AND PLANT

(71) Applicant: Cemvita Factory, Inc., Houston, TX (US)

(72) Inventors: Zachary Richard BROUSSARD, Houston, TX (US); TJ TIDWELL, Houston, TX (US); Michael SAMUEL, Houston, TX (US); Edward John KOCH, Houston, TX (US); Raja Subramanian RAMANATHAN, Houston, TX (US); Renata Amanda GONÇALVES, Houston, TX (US); Aaron TREVINO, Houston, TX (US); Addien WRAY, Houston, TX (US); Bárbara de Freitas MAGALHÃES, Houston, TX (US)

(21) Appl. No.: 18/618,868

(22) Filed: Mar. 27, 2024

Related U.S. Application Data
(60) Provisional application No. 63/492,624, filed on Mar. 28, 2023.

(30) Foreign Application Priority Data
Apr. 18, 2023 (GB) 2305672.4

(51) Int. Cl. C12P 3/00; C12P 39/00

(52) U.S. Cl. CPC C12P 3/00; C12P 39/00

(57) ABSTRACT

The invention concerns process and plant for the microbial production of hydrogen from the site of hydrocarbonaceous deposit, the process comprising modifying the composition of the deposit through the introduction into or into the vicinity of the deposit of at least one hydrogen producing microorganism, wherein the at least one hydrogen producing microorganism or at least one microbiological condition conducive to the thriving of the microorganism is selected, or its selection is aided, by means of continuous flow methodology, the plant comprising means in the form of continuous flow methodology apparatus for selecting at least one hydrogen producing microorganism and/or for selecting at least one microbiological condition conducive to the thriving of the microorganism; and means for supplying the at least one hydrogen producing microorganism into the hydrocarbonaceous deposit.

This application claims priority to U.S. Provisional Application No. 63/492,624, filed Mar. 28, 2023 and to GB Application No. 2305672.4, filed Apr. 18, 2023. The entirety of the aforementioned applications are incorporated herein by reference.

### FIELD

The present application concerns a process and plant for the microbiological production of hydrogen from a hydrocarbonaceous deposit, especially a subterranean deposit, employing continuous flow methodology, for example core flood and/or sandpack methodology, for the purposes of selecting one or more hydrogen-producing microorganisms for delivery into or into the vicinity of the hydrocarbonaceous deposit.

### BACKGROUND

Hydrogen is an important fuel and chemical process substrate. It is known in the art to use microbes to produce hydrogen from hydrocarbon substrates.

WO2005115648 describes a process for characterizing and then manipulating the environment of fermentative syntrophic microorganisms naturally present in a petroleum-bearing subterranean formation in order to promote microbial generation of hydrogen in the formation.

WO2015052806 similarly describes the use of an Fe(III) activator compound to stimulate subterranean microbial hydrogen and methane and also suggests ex situ cultivation and subsequent re-injection of microbes naturally occurring in the subterranean environment.

WO2005113784 describes a method for enhancing microbial production of hydrogen from a hydrocarbon rich deposit. The disclosure favors achieving this by stimulating the metabolic activities of indigenous microorganisms within the deposit, including by the introduction of exogenous (possibly genetically modified) organisms having metabolic capabilities of interest. These metabolic capabilities are not defined except insofar as their impact is to improve net hydrogen production, and contextually this seems to mean by inhibiting the consumption of hydrogen rather than by metabolization of hydrocarbons to hydrogen within the deposit. This document therefore fails to appreciate or to disclose the introduction into the deposit of further microorganisms which are non-native to the deposit and which themselves are capable of metabolizing hydrocarbons to molecular hydrogen and which serve to increase hydrogen production in the deposit by positively diversifying the microbiological abundance of microorganisms in the deposit.

WO0234931 describes a method of generating and recovering methane from solid carbonaceous deposits. This disclosure suggests to inject bacterial consortia into such deposits and recognizes that hydrogen as well as methane may be produced, but methane production is the clear objective of the disclosure and fermentative hydrogen producers are envisioned as being useful only insofar as they provide a feedstock for methanogenesis.

Our co-pending application PCTUS2022076925, the contents of which are hereby incorporated by reference, describes a process for the microbiological production of hydrogen from a hydrocarbon-rich deposit. The process comprises modifying the composition of the deposit by the introduction of hydrogen producing microorganisms selected to diversify the microbiological abundance of hydrogen-producing microorganisms in the deposit and for the preferential production of hydrogen over methane.

WO2011098770 describes a method and a system for predicting the effect of microbes injected into an oil-bearing reservoir using computer-implemented modeling based on simulating the effect of injecting microbes into a multiple core flood apparatus.

Singh et al., “Overview of Carbon Capture Technology: Microalgal Biorefinery Concept and State-of-the-Art”, Frontiers in Marine Science, 6, 2019, details an overview of carbon capture technology, and in particular microalgal biorefineries, as a means to combat climate change. Singh et al. detail how microalgae can be used to convert raw materials into high and low value products and fuels derived from biomass.

Barnhart et al., “Enhanced coal-dependent methanogenesis coupled with algal biofuels: Potential water recycle and carbon capture”, International Journal of Coal Geology, 171, 2017, 69-75, details methods for stimulating the production of methane from coal bed methanogenesis by introducing further additives to the coal bed to stimulate the activity of the native microorganisms.

Davis', “Organic amendments for enhancing microbial coalbed methane production”, Montana State University, 2017, details the use of organic amendments, i.e. the addition of microbes and/or additives, to enhance the microbial processes for coal-to-methane produced coalbed methane, a form of natural gas found in subsurface coal beds wherein the methane is generated by native microbes to the coal bed. The process detailed therefore focuses on the addition of additives to enhance an already natural process.

Veshareh et al “The light in the dark: In-situ biorefinement of crude oil to hydrogen using typical oil reservoir Thermotoga strains”, International Journal of Hydrogen Energy, Volume 47 (8), 2022 (https://doi.org/10.1016/j.ijhydene.2021.11.118), details the use of bacteria extracted from hydrocarbon reservoirs with the use of native bacteria such as Thermotoga strains, to convert hydrocarbons into hydrogen.

The process detailed in this paper uses complex nutrients (including non-hydrocarbon nutrients) in all conditions that were investigated. The organisms used in the study may themselves grow, ferment and produce hydrogen using the non-hydrocarbon nutrients present. Therefore, this paper fails to appreciate or disclose cell growth and the production of hydrogen without the presence of additional fermentative compounds, such that the results obtained in the paper are not directly attributed to hydrocarbon-specific fermentation. Additionally, there is no consideration in the paper towards the introduction into the deposit of further microorganisms which are non-native to the deposit.

In the exemplified prior art examples, the primary focus concerns the manipulation of indigenous microbial populations or their environment, in some cases with the aid of other microbes which inhibit hydrogen consumption or which are themselves methanogenic. Little attention has been paid to the practical difficulties of selecting on-site microbiological reagents, especially in circumstances where it is desirable to select, adapt or engineer such reagents for optimization of their hydrogen producing efficacy at the site in question.

The use of continuous flow methodology, such as core flood and/or sandpack methodology, are known in the art.

US2012/0255726 describes a method for in-situ oxygen generation in an underground hydrocarbon containing formation. This document discloses that an optional middle phase could be to verify the chance of success by core flood experiments. This document is not concerned with the production of hydrogen.

U.S. Pat. No. 8,868,392 describes a method for predicting the effect of microbes injected into an oil-bearing reservoir which is capable of being represented by a first computer-generated grid including a first plurality of grid blocks.

U.S. Pat. No. 8,658,412 describes methods wherein microorganisms are used to alter the interface of hydrocarbons and hydrocarbon-coated surfaces to increase oil recovery.

U.S. Pat. No. 7,484,560 describes a microbial consortium containing three hyperthermophilic, barophilic, acidogenic, anaerobic bacterial strains for enhanced oil recovery. This document discloses the use of core flood study for the in situ application of a consortium for enhanced oil recovery.

US2018/0237683 describes the microbial production of methane from carbonaceous media.

In the exemplified prior art examples disclosing continuous flow methodology, dynamic high-pressure tests are provided with no consideration or disclosure towards the microbial production of hydrogen (or how this may be improved), especially in circumstances where it is desirable to select, adapt or engineer such reagents for optimization of their hydrogen producing efficacy at the site in question.

Additionally, there is no consideration provided in the prior art towards processes related to the dynamic injection of nutrients, additives, microbes into porous media at high temperature conditions to produce gases, in particular hydrogen.

There has therefore been little to no consideration in the art for providing a process that utilizes dynamic porous media for continuous flow methodology, such as core flood and/or sandpack methodology, combined with analysis to allow selection of the most effective microorganism at representative—in at least one of its geochemical or geomineralogical characteristics—of on-site material. Thus, there remains a need in the art for a process which addresses the problems associated with prior art.

### SUMMARY

According to a first aspect of the present application there is provided a process for the microbial production of hydrogen from the site of hydrocarbonaceous deposit, the process comprising modifying the composition of the deposit through the introduction into or into the vicinity of the deposit of at least one hydrogen producing microorganism, wherein the at least one hydrogen producing microorganism or at least one microbiological condition conducive to the thriving of the microorganism is selected, or its selection is aided, by means of continuous flow methodology.

Continuous flow methodology is to be construed as a dynamic and continuous methodology that includes a continuous (or semi-continuous) flow throughout the hydrogen production process i.e., feeding a microbial mixture through a sample of hydrocarbonaceous deposit and analysing the output on a continuous (or semi-continuous) basis such that physiological parameters can be adjusted in response to the output.

The continuous flow methodology may be for example core flood and/or sandpack methodology. The inventors have advantageously found that a continuous (or semi-continuous) flow methodology provides a means to simulate the environmental characteristics of one specific environment (i.e., hydrocarbonaceous deposit) in another environment and provides a greater control of physiological parameters (for example, continuous feed supply, temperature, pH, salinity, pressure and/or anaerobic atmosphere).

This is in contrast to static or batch methodology where there is no mid-process analysis and thus no control of physiological parameters, therefore replication of environmental characteristics of a specific environment is difficult to achieve.

The continuous flow methodology (i.e., core flood and/or sandpack methodology) may comprise extracting a material sample from the site or selecting a material sample representative of a material from the site and subjecting the sample to analysis determinative of its response to microbiological conditions.

The phrase “into the vicinity” is to be construed as the at least one hydrogen producing microorganism being introduced in fluid communication with the hydrocarbonaceous deposit.

The phase “or at least one microbiological condition conducive to the thriving of the microorganism” is to be construed as any suitable condition where the microorganism is capable of metabolizing hydrocarbonaceous materials to molecular hydrogen. Examples of hydrocarbonaceous materials may include, but are not limited to, sugars, starches and/or credential components of crude oil. Examples of suitable conditions may include a pH of between about 5-8, a temperature of between about 120-180° F. (about 48-82° C.), pressure between about 15 psi-2000 psi (about 1 atm to 136 atm), salinity of between about 0-6% by wt, phosphate sources between about 50 μmol-5 mmol and/or nitrogen sources between about 50 μmol-5 mmol.

The continuous flow methodology (i.e., core flood and/or sandpack methodology) methodology advantageously may study how the sample changes over a period of time, for example over a time of days, weeks, several months or year(s), thereby reflecting the changes in spatial and temporal biogeochemical environments in real world situations. This inventors have found that this advantageously results in the selection, adaption or engineering of reagents for optimising the hydrogen producing efficacy at the site in question. This level of analysis cannot be achieved with short-term static tests.

Preferably the sample extraction or sample selection is conducted under anaerobic conditions. Preferably the extracted or selected sample is then maintained under anaerobic conditions in any interim period between sample extraction or selection and its subjection to continuous flow methodology (i.e., core flood and/or sandpack methodology), which also preferably takes place under anaerobic conditions. These precautions can help to ensure that the microbiological material(s) extracted from the site or otherwise selected are not degraded, or not excessively degraded, following extraction or selection and during exploratory investigation by means of continuous flow methodology.

The material sample for continuous flow methodology is a porous medium. The porous medium may be sandstone rock, clay, shale rock, sand, igneous rock or metamorphic rock.

The material sample continuous flow methodology, for example core flood methodology, may comprise a geological or mineralogical sample (for example rock, porous rock, sand, shale, clay) and/or a hydrocarbonaceous sample (for example oil, natural gas or coal). The geological or mineralogical sample may be extracted from the site of the hydrocarbonaceous deposit or it may be otherwise selected to be representative—in at least one of its geochemical or geomineralogical characteristics-of on-site material. For example, when the site is characterisable by the geological presence of sandstone material the process of the invention may be realized by selecting a standard geological or geomineralogical core sample—such as Berea sandstone—and subjecting that selected sample to analysis by means of core flood methodology in order to determine suitable conditions for the sustainability or enhancement of microbiological activity with respect to hydrogen production of hydrogen-producing microorganism(s) within the sample. The material sample for sandpack methodology may be sand.

The material sample may be subjected to determinative analysis on or off the site of the hydrocarbonaccous deposit.

The determinative analysis may comprise maintaining the sample under desirable conditions of temperature, pressure and/or chemical environment (e.g. salinity) and determining a response of the sample to one or more microbiological conditions.

The desirable conditions may at least to some extent replicate one or more of the those conditions (of temperature, pressure and/or chemical environment) of the site location from which the sample is extracted.

The at least one hydrogen producing microorganism may be a non-native microorganism. By “non-native” is meant that the microorganism does not occur naturally at the site, or does not occur naturally in abundance at the site. An “abundance” may be defined as comprising, in a sample taken from the site, over 10%, over 5% or over 1% w/w of all microorganisms in the sample.

The hydrogen producing microorganism or at least one microbiological condition conducive to the thriving of the microorganism may be selected by means of the continuous flow methodology positively to diversify the microbial abundance of hydrogen-producing microorganisms in the deposit when charged thereto, and optionally also for the preferential production of hydrogen over methane. This will especially be the case when the hydrogen producing microorganism is a non-native hydrogen producing microorganism.

According to a second aspect of the present application there is provided plant for the microbial production of hydrogen from a hydrocarbonaceous deposit comprising: means in the form of continuous flow methodology (for example core flood and/or sandpack methodology) apparatus for selecting at least one hydrogen producing microorganism and/or for selecting at least one microbiological condition conducive to the thriving of the microorganism; and means for supplying the at least one hydrogen producing microorganism into the hydrocarbonaceous deposit.

Preferably the means for supplying the at least one hydrogen producing microorganism into the hydrocarbonaceous deposit comprise means for also providing into the deposit the at least one microbiological condition conducive to the thriving of the microorganism.

The continuous flow methodology apparatus may be provided on or off the site of the hydrocarbonaceous deposit.

The plant may additionally comprise means for extracting from the hydrocarbonaceous deposit a product stream comprising at least hydrogen gas generated by the microbiological action in the deposit of the at least one hydrogen producing microorganism selected by means of continuous flow methodology, for example core flood and/or sandpack methodology.

The continuous flow methodology may be used to identify the most suitable microorganisms for the deposit, which therefore improve the efficiency of the overall process and improve the yields of hydrogen produced for the deposit.

The plant provides an effective means of converting hydrocarbon deposits in situ. Once a target well or other site containing such hydrocarbon deposits has been identified its geological and/or geochemical characteristics and/or its responsiveness to microbiological conditions are assessed by means of continuous flow methodology. In this manner a suitable microbiological agent (or a consortia of microbial agents) may be identified for the most efficacious extraction of hydrogen from that particular site.

In one aspect of the invention the at least one microbiological agent(s) identified by means of continuous flow methodology (for example core flood and/or sandpack methodology) may be charged to a transportable microbiological incubator, optionally together with suitable nutrients and/or adjuvants. The incubator may be maintained under conditions effective for functional maintenance of the microbiological agent(s), and transported in such condition to the site whereupon it can be established by means of suitable connective infrastructure as a “plug and play” unit feeding the microbiological agent(s) and any associated materials directly into or into the vicinity of the hydrocarbon deposits, this being the subject of our co-filed application P13140US.

The present application provides a significant advantage over the existing state of the art because continuous flow analysis allows the response of the geological material to treatment with different microorganisms and nutrient conditions with regards to the efficacy of hydrogen generation from the material to be effectively explored, improving the overall efficiency of a hydrogen extraction process. Through integration continuous flow analysis with a process converting a hydrocarbonaceous deposit to hydrogen using microorganisms, a more efficient and versatile conversion of hydrocarbon deposits to hydrogen can be achieved.

### BRIEF DESCRIPTION OF THE DRAWINGS

The invention will now be more particularly described with reference to the following examples and figures, in which;

FIG. 1 is a schematic flow diagram of a process operating in accordance with one aspect of the invention.

FIG. 2 is a schematic illustration of a core flood analysis system suitable for use with the process of FIG. 1.

FIG. 3 is a schematic illustration of a sandpack analysis system suitable for use with the process of FIG. 1; and

FIG. 4a is a schematic illustration of a production system manifold and FIG. 4b is an illustration of an injection system manifold suitable for us with the process of FIG. 1.

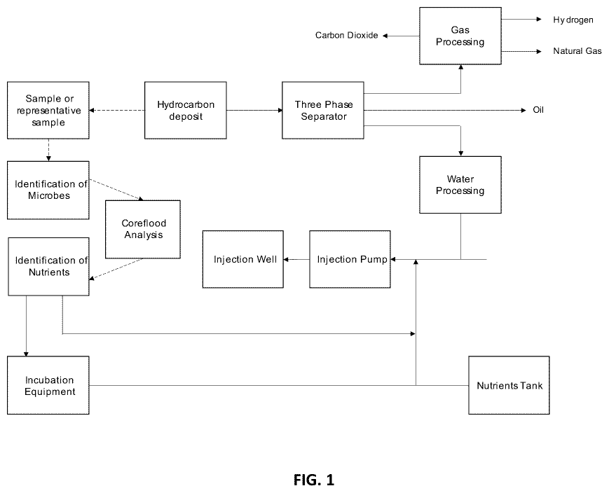

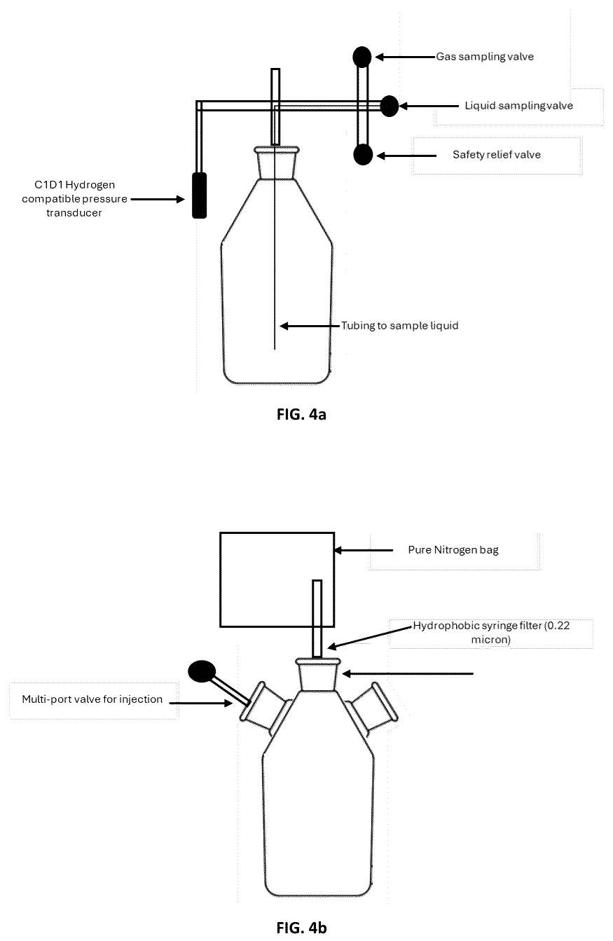

The plant, as illustrated by FIG. 1, comprises:
a. a hydrocarbonaceous deposit;
b. means for extracting a material sample from the deposit or selecting a material sample representative of a material from the site;
c. continuous flow methodology (i.e., core flood or sandpack methodology) apparatus for determining the responsiveness of that sample to a variety of physical, chemical and/or microbiological conditions;
d. equipment for incubating at least one microorganism selected by means of the core flood methodology for efficacious hydrogen production from the deposit;
e. means for supplying the selected microorganism into the hydrocarbonaceous deposit; and
f. means for extracting produced hydrogen from the deposit.

The hydrocarbon rich deposit may contain crude oil, natural gas or coal. The deposit may comprise a porous rock formation.

The three phase separator separates water, oil and gas components collected from the hydrogen-rich deposits. The gaseous components are collected and are provided to a gas processing station. The liquid hydrocarbon components are collected and may be subject to further processing, or sold as an end product. The aqueous components are collected and may be provided to a water processing unit, or disposed of.

The gas processing station is used to process the gaseous products collected from a deposit, and may be used to separate hydrogen, natural gas, and carbon dioxide. In some embodiments of the present application, the carbon dioxide may be collected usefully re-processed. The gas processing station also provides a means for cleaning the product gases for further use, or for sale as an end product.

The water processing unit may be responsible for cleaning the water collected by the three phase separator. In some embodiments of the present application, undesirable volatile fatty acids (VFAs) may be removed in the water processing unit. The water collected by the three phase separator may further be used as a supply to the injection pump. An advantage of recycling the water from the extraction stages is that waste water is reduced.

The incubation equipment contains the at least one hydrogen producing microorganism capable of modifying the composition of the hydrogen-rich deposit. The incubation equipment may comprise at least one bioincubator responsible for producing the hydrogen producing microorganisms. In some embodiments, the incubation equipment may be supplied with water from the water processing unit.

The nutrient tank may contain the nutrients or adjuvants required by the hydrogen producing microorganisms. In preferred embodiments of the present application, the incubation equipment and the nutrient tank may be housed together.

The flow of microbes, nutrients from the incubation equipment and water passes to an injection pump, which injects the reaction mixture into an injection well, which is the deposit wherein the microbial production of hydrogen from a deposit occurs.

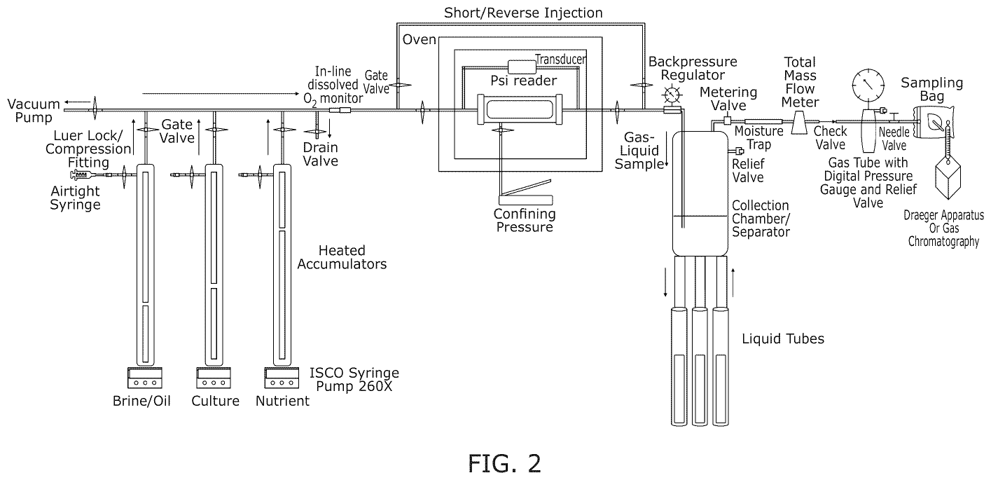

The core flood methodology is described more particularly below in Example 3 with reference to FIG. 2.

For the avoidance of doubt, all features relating to the continuous flow methodology, for example core flood methodology is also related, where appropriate, to sandpack methodology of the present application and vice versa.

For the avoidance of doubt, all features relating to the method of the present application also relate, where appropriate, to the plant of the present application and vice versa.

It should be apparent that any of the embodiments of the invention, and each or any of their described variants, may be provided in combination with the first or second aspects of the invention and each or any of its described variants and/or in combination with any one or more of each other.

### DETAILED DESCRIPTION

In the process and plant of the invention the assessment of the geological, geochemical and/or geo-biochemical characteristics, in particular the response of the geological material to treatment with different microorganisms and nutrient, adjuvant or salinity conditions with regards to the efficacy of hydrogen generation from the material, is effected by means of continuous flow analysis.

The technique of continuous flow methodology, for example core flooding, is a method wherein a hydrocarbonaceous and/or mineralogical sample from a given location, or a comparable selected material, is placed under in situ reservoir conditions at least to some extent replicative of those occurring at the given location, and the responsiveness of the sample to a variety of reservoir conditions is explored under laboratory conditions. So, for example, the technique may comprise a method where a sample of a hydrocarbon or rock deposit is removed from a subterranean reservoir or an outcrop, and is then placed under the same in situ reservoir conditions for testing in a laboratory setting. Single continuous flow methodology such as core flood and/or sandpack experiments are well known in the crude oil recovery industry and are analysed in order to give an indication of the effect of reservoir treatments at the laboratory scale.

In continuous flow methodology analysis such as core flood and/or sandpack analysis a geological sample from the site is maintained, in the core, under conditions of high pressure and temperature replicative of the in situ conditions of the hydrocarbon deposit. The response of the sample to different microbial populations and conditions may be assessed and the most suitable reagents determined for a given deposit. Once that determination has been made the microbial reagents and associated nutrient or adjuvant package can be injected into a hydrocarbon deposit. In the present application this technique is used to predict the effect of microorganisms injected into an hydrocarbon deposit.

In some embodiments of the present application, the selected microorganisms may be charged to a mobile fermentation unit and shipped to site for injection into the deposit.

The continuous flow analysis may be carried out at a high pressure, to mimic the in situ conditions of the deposit. The continuous flow methodology may be carried out at pressures of, up to 2,000 psi, up to 4,000 psi, up to 6,000 psi, up to 8,000 psi, or in most preferable embodiments up to 10,000 psi.

Continuous flow analysis also allows for the determination of the propagation behavior of microbes in porous media, which will give us some insight on field implementation. This testing is important for risk mitigation as well as for inching closer to testing under real world conditions.

The continuous flow analysis system of the present application comprises the fluid injection, core flood testing, sandpack testing, and fluid analysis.

The system assesses how high-pressure high-temperature (HPHT) conditions affect fermentation through analysis of factors such as reaction behavior, rates, volumes, side products and precipitation. Continuous flow methodology, for example core flood analysis and/or sandpack also assess the efficiency of the culture in propagating through long distances within the rock formation, and whether cultures can be recycled. More broadly, core flood analysis also allows the assessment of fluid flow through pores, and the effect on flow on the efficiency of fermentation.

As continuous flow analysis permits the assessment of the in situ conditions of a hydrocarbon deposit, it informs how the conditions are scaled up to provide full field production metrics, including reserves, production rates and best injection scenarios.

In the fluid injection stage, fluids are maintained under anaerobic conditions. The fluid injection system may comprise multiple heated accumulators containing brine, oil, microorganism cultures, and/or nutrients. The heated accumulators may be connected to a common line to the core flood chamber by gate valves to regulate the flow to the core flood. Vacuum pumps and airtight syringes may be used to add anaerobic microbes into pump systems. It is important that the dead volume is limited and that connections are easily replaceable. A dissolved oxygen monitor may be used to detect oxygen levels within injection system to ensure fully anaerobic conditions. The system may comprise a multi-switch valve, the inventors have found that a multi-switch valve facilitates the injection. The system may further comprise a drain valve. The system may further comprise a vacuum pump to maintain the system under vacuum. The vacuum may help in removing any trace oxygen from the system, and can be replaced with inert gases, for example nitrogen.

In some embodiments, the flow range may be between 0.001 and 1 ml/min, 0.001 and 10 ml/min, or 0.001 and 107 mL/min.

In a further particular embodiment, the maximum vacuum pump pressure may be about −1 bar (−14.7 psig or 0 atm).

The container used to contain the fluid injection media in the fluid injection stage may be a three necked container, optionally wherein one of the necks is used to provide pressure equalization within the container. The container may be made from any material that minimizes hydrogen embrittlement and prevents hydrogen leakage, for example glass, stainless steel or titanium alloy.

In the continuous flow testing stage, for example the core flood testing stage, a core holder is contained within an oven, and may be made of HASTELLOY C276, a Nickel-chromium-molybdenum wrought alloy that is highly corrosion resistant, stainless steel or titanium alloy, which minimizes or eliminates hydrogen embrittlement. The core holder holds the core, in which the sample is contained. In some embodiments the core holder may hold 12 in. length and 1.5±0.1-inch diameter cores. The fluids may be injected into the core, and subjected to high-temperature and pressure conditions. The core holder may comprise a multiplicity of taps across the length of the core holder, thereby allowing in-situ evaluation of the sample, for example, the analysis of microbial fermentation process parameters and permeability and absorption measurements of the process. The core holder may be tri axial, which advantageously simulates a real world situation such that the user may apply both confining and axial stresses to the porous media. Following a core flood process, the fluid samples pass through a line, which may be controlled by valves, to the fluid sampling and analysis stage.

In the sandpack testing stage, there would be no confining or axial pressure applied to a sandpack bioreactor, thereby the testing stage is considered to be analogous to the core holder in a core flood testing stage.

In the fluid analysis stage, the gas-liquid sample from the core passes unto a liquid separator collection chamber, which may further comprise liquid collection tubes. The collection chamber collects all gas for discrete or continuous measurements. The system may be connected to a gas chromatograph to allow continuous gas analysis. The liquid collection tubes may be used to trap liquids under anaerobic conditions for further analysis.

Samples are collected from the collection chamber or separator and pass through a moisture trap to sampling bags. Samples may be analysed by a Draeger apparatus or by gas chromatography to assess different parameters. Preferably, the analysis is dynamic analysis of the sample. In some embodiments the measurements made are; liquid volumes (oil/water) vs time, gas flow rate and cumulative mass, the concentration of ATP or side products, pH, gas composition (batch/continuous), the culture adsorption on rocks, and/or emulsion or phase separation. Various analysis of the samples may include, but is not limited to, microbial adenylate charge testing (i.e., AMP/ADP/ATP testing), DNA sequencing, adaptive lab evolution, biogeochemical analysis of injected and effluent media, gas compositional analysis, redox changes, organic acid concentrations, pH changes, hydrogen to carbon dioxide mole ratio, bioenergetics including changes in Gibbs energy and enthalpy, total petroleum hydrocarbon analysis, crude oil compositional changes (via Orbitrap gas chromatography mass spectroscopy or Fourier Transform-Ion cyclotron resonance), SARA (saturates, aromatics, resins, asphaltenes) analysis, and/or PIANO (paraffins, isoparaffins, aromatics, naphtenes, olefins) analysis.

In some embodiments, the continuous flow methodology porous media may be broken down to conduct sessile microbial fermentation analysis.

The combination of analysis and data processing of the different parameters has advantageously been found to provide a quantitative description of microbial metabolic processes involved in hydrocarbon fermentation and/or hydrogen production.

The hydrogen production and/or hydrocarbon fermentation process may affect the fluid and/or porous media transport processes of the continuous flow methodology. For example, at least one of reservoir pressure, hydrogen relative permeability, hydrogen adsorption, fingering effects, capillary pressure, hydrogen and crude oil phase behaviour may be affected. The inventors of the present application have advantageously found that continuous flow methodology provide the means to choose the most suitable microbiological agent (or a consortia of microbial agents) for the most efficacious extraction of hydrogen from that particular whilst minimizing any potential damage to the reservoir.

In some embodiments, metering valves may be used to regulate flow. In further embodiments, moisture traps may be added to remove humidity and obtain more accurate measurements.

The inventors have surprisingly found that the combination of analysis and data processing of the invention yields a quantitative description of the microbial metabolic processes involved in hydrocarbon fermentation and hydrogen production, such that the inventors may predict the magnitude of microbially produced hydrogen under varied geological and geochemical conditions.

The hydrogen producing microorganism may be:
a. a microorganism not naturally present in the hydrocarbonaceous deposit; and/or
b. of a strain of microorganisms not naturally present in the hydrocarbonaceous deposit; and/or
c. of a species of microorganisms not naturally present in the hydrocarbonaceous deposit; and/or
d. of a genus of microorganisms not naturally present in the hydrocarbonaceous deposit;
e. a microorganism naturally present in the hydrocarbonaceous deposit; and/or
f. a microorganism naturally present in the hydrocarbonaceous deposit but genetically modified to increase (relative to the naturally present microorganism) its propensity for hydrogen production by the metabolization by that microorganism of one or more hydrocarbons contained within the deposit.

The at least one hydrogen producing microorganism may be one of a plurality of different hydrogen producing microorganisms, strains of microorganisms, species of microorganisms, genera of microorganisms and/or naturally occurring but genetically modified organisms introduced into the deposit. Genetic manipulation of microorganisms naturally present in the deposit to form non-native species may be effected by directed evolution or other form of synthetic biology. The plurality may be greater than two, greater than three, greater then four, greater than five and/or greater than ten.

The hydrogen producing microorganism(s) may have a propensity to metabolize one or more hydrocarbons contained within the deposit to molecular hydrogen (preferably in preference to methane) such that the yield of production of molecular hydrogen (H₂) from the metabolization is higher than the yield of production of methane by at least 1%, by at least 10%, by at least 100% and/or by at least 1000%.

The hydrogen producing microorganism(s) may be introduced into the deposit and accompanied during, after or upon its introduction by at least one nutrient selected to promote the growth of said microorganism and introduced into the deposit for that purpose, optionally wherein the nutrients are supplied from a nutrient reservoir.

The at least one nutrient may be selected, or its selection aided, by means of continuous flow methodology, for example core flood and/or sandpack methodology.

The at least one nutrient may be selected preferentially to promote the growth of the said microorganism in preference to at least one, to at least some or to all of any native microorganisms in the deposit.

The nutrient may comprise one or more of:
a. one or more salts selected from:
   i. phosphates; and/or
   ii. halides; and/or
   iii. nitrates, ammonium salts, nitrogenous salts; and/or
b. one or more carbohydrates selected from:
   i. sugars; and/or
   ii. starches; and/or
c. one or more vitamins; and/or
d. complex nutrients, optionally comprising yeast extracts; corn steep liquor; biomass; bacterial an/or algal biomass.

As will be apparent from Example 1 below it is particularly advantageous to include at least one carbohydrate and/or complex nutrient in the at least one nutrient.

The hydrogen producing microorganism may be introduced into the deposit and accompanied during, after or upon its introduction by at least one pH regulator selected to regulate the pH environment in which the microorganism resides in the deposit and introduced into the deposit for that purpose. The pH regulator may be selected to regulate the pH of the hydrogen producing microorganism environment in the deposit to a pH within the range of from about 5 to about 9, from about 6 to about 8 and/or from about 6 to about 7.

The pH regulator may optionally also serve as a nutrient—for example, phosphate can acts as both a nutrient and as a buffering agent.

The hydrogen producing microorganism may be introduced into the deposit and accompanied during, after or upon its introduction by at least reducing agent which may or may not be included as part of the nutrient package. Suitable reducing agents include thioglycolic acid (and salts such as sodium thioglycolate), cysteine HCl, Na2S, FeS, dithiothreitol, sodium dithionite, ascorbic acid, oxalic acid, sodium sulfite, sodium metabisulfite, 2-mercaptoethanol, sodium pyruvate, glutathione and compatible mixtures of two or more thereof.

The hydrocarbonaceous deposit is preferably a liquid hydrocarbonaceous deposit, e.g oil/bitumen/heavy oil.

The at least one hydrogen producing microorganism may have a genus of Syntrophobacter, Syntrophus, Syntrophomonas, Thermoanaerobacter, Thermotoga, Pseudothermotoga, Thermoanaerobacterium, Fervidobacterium, Thermosipho, Haloanaerobium, Acetoanaerobium, Anacrobaculum, Geotoga, Petrotoga, Thermococcus, Pyrococcus, Clostridium, Enterobacter, Klebsiella, Ethanoligenens, Pantoca, Escherichia, Bacillus, Caldicellulosiruptor, Pelobacter, Caldanacrobacter, Marinitoga, Oceanotoga, Defluviitoga, Kosmotoga, Caloranacrobacter or a combination or mixture thereof.

The hydrogen producing microorganism may express at least one protein selected from hydrogenases, dehydrogenases, hydroxylases, carboxylases, esterases, hydratases and acetyltransferases having an amino acid sequence at least 95% identical to a sequence expressed by an upregulated or downregulated gene selected from mth (EC 1.12.98.2), mrt, hycA (ID: 45797123), fdhF (ID: 66346687), fh1A (ID: 947181), ldhA (ID: 946315), nuoB (ID: 65303631), hybO (ID: 945902), fdh1, narP, ppk or Pepc by expressing a non-native protein expressing nucleotide sequence, wherein an amount of hydrogen produced or protein produced by the hydrogen producing microorganism is greater than that produced relative to a control microorganism lacking the non-native protein expressing nucleotide sequence.

The hydrogen producing microorganism may be a recombinant microorganism.

The recombinant microorganism may express at least one Coenzyme M reductase and or dehydrogenase protein having a gene sequences at least 95% identical to SEQ ID NO. [mmg: MTBMA_c15480], [mth: MTH_1015], [mmg: MTBMA_c15520], [mmg: MTBMA_c15490], [mth: MTH_1166], [mth: MTH_1167], [eco: b4346], [eco: b4345], [ag: AAA22593], [mea: Mex_Ip4538, [mea: Mex_1p4535], [ag: ACS29499], [ag: CAH55641], [mrd: Mrad2831_0508], by expressing a non-native Coenzyme M reductase and or dehydrogenase expressing nucleotide sequence.

Preferably, an amount of hydrogen produced or protein produced by the hydrogen producing microorganism is greater than that produced relative to a control microorganism lacking the non-native protein expressing nucleotide sequence.

The environment of the hydrocarbonaceous deposit and the introduced hydrogen producing microorganism may constitute an enclosed bioreactor, being a bioreactor subterranean formation, a bioreactor landfill enclosure, or a combination thereof.

In this case there is provided in accordance with the aforesaid first aspect of the invention and any or each of its described variants a method of increasing hydrogen production from an enclosed bioreactor (as constituted by the environment of the hydrocarbonaceous deposit and the introduced hydrogen producing microorganism) comprising: providing a baseline reaction mixture in the enclosed bioreactor, wherein the baseline reaction mixture includes a hydrocarbon, water, and a baseline amount of at least one microorganism; producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture; measuring a baseline amount of hydrogen in a baseline gas sample of gasses collected from the enclosed bioreactor; increasing hydrogen production from the enclosed bioreactor by forming a synthetic reaction mixture including at least one hydrogen producing microorganism selected by means of core flood methodology, and harvesting the hydrogen from the enclosed bioreactor at a hydrogen harvesting rate by separating the hydrogen from other gasses and transferring the hydrogen into a hydrogen storage container.

The synthetic reaction mixture is formed by: adding at least one hydrogen producing microorganism selected by means of continuous flow methodology until a percentage of the hydrogen producing microorganism in the synthetic reaction mixture is at least 20% of a total amount of microorganisms in the synthetic reaction mixture.

The method may further comprise after providing the baseline reaction mixture, but before forming the synthetic reaction mixture, producing baseline environmental data from the baseline reaction mixture. The baseline environmental data may include one or more of the following measurements of a baseline environmental sample from the baseline reaction mixture: pH; temperature; water analysis; oxidation-reduction potential; pressure; dissolved oxygen; hydrocarbon concentrations; volatile fatty acids concentrations; cation concentration; anion concentration; concentration of gases (such as one or more of NH₃, CO₂, CO, H₂, H₂S and CH₄); salt concentration; and metal concentration. Continuous flow methodology may be used to investigate any one or more of these parameters.

The baseline microorganism sample and the baseline environmental sample may be the same or different.

The hydrogen harvesting rate may be at least about 0.1 L/hr, or at least about 1 L/hr, or at least about 10 L/hr, or at least about 100 L/hr. The hydrogen harvesting rate may be up to about 10⁶ L/hr, or up to about 10⁵ L/hr, or up to about 10⁴ L/hr, or up to about 10³ L/hr. The hydrogen harvesting rate may be from about 0.1 L/hr to about 10⁶ L/hr, or from about 0.1 L/hr to about 10³ L/hr, or from about 10³ L/hr to about 10⁶ L/hr.

The subterranean formation may include a natural formation, non-natural formation, a hydrocarbon-bearing formation, a natural gas-bearing formation, a methane-bearing formation, a depleted hydrocarbon formation, a depleted natural gas-bearing formation, a wellbore, or a combination thereof.

The bioreactor landfill enclosure may include a landfill that is enclosed by a building material. The building material may include at least one of a brick, a cement, a plastic, a non-natural rubber, a geomembrane of any kind, concrete, steel, a glass, or a combination thereof.

The process may comprise manipulating the subsurface of the hydrogen producing microorganism or microbial communities through supplying chemical additives (for example, nitrate, 2-bromoethanesulfonate (2-BES) and/or surfactants) and/or the introduction of multiple microbial strains, for example. The inventors have advantageously found that such manipulation may concurrently produce and retrieve hydrogen, whilst preventing the production of unwanted and dangerous by-products, for example hydrogen sulfide.

The hydrogen producing microorganism may be supplied to the deposit in combination with compounds that inhibit the growth of undesirable microorganisms, for example the inclusion of nitrate to downregulate sulfur-reducing metabolisms.

In some embodiments, the hydrogen producing microorganisms may be supplied to the deposit in combination with non-fermentative microbes. The inventors have found that the combination of the hydrogen producing microorganisms and non-fermentative microbes simultaneously emulsify hydrocarbon compounds in situ, thereby increasing the physical contact of fermenting microbes and hydrocarbons, and removing acids (for example acetic acid produced by hydrocarbon fermentation), thereby increasing the thermodynamic favorability of the metabolism and increasing hydrogen production.

The hydrogen producing microorganism may be supplied to the deposit in combination with a hydrogen production enhancer, for example a biocidal inhibitor, a methanogenesis inhibitor, a sulfate reduction inhibitor, a nitrate reduction inhibitor, an iron reduction inhibitor, or any suitable combination thereof.

The biocidal inhibitor may be glutaraldehyde, a quaternary ammonium compound, formaldehyde, a formaldehyde releaser such as 3,3′-methylenebis[5-methyloxazolidine], dibromonitrilopropionamide, tetrakis hydroxymethyl phosphonium sulfate, chlorine dioxide, peracetic acid, tributyl tetradecyl phosphonium chloride, methylisothiazolinone, chloromethylisothiazolinone, sodium hypochlorite, dazomet, dimethyloxazolidine, trimethyloxazolidine, N-bromosuccinimide, bronopol, or 2-propenal, or a mixture thereof.

The methanogenesis inhibitor may be bromethane sulfonic acid, an aminobenzoic acid, 2-bromoethanesulfonate, 2-chlorocthanesulfonate, 2-mercaptoethanesulfonate, lumazine, a fluoroacetate, nitroethane, or 2-nitropropanol, or a mixture thereof.

The sulfate reduction inhibitor may be a molybdate salt, a nitrate salt, a nitrite salt, a chlorate salt, or a perchlorate salt or a mixture thereof.

The nitrate reduction inhibitor may be sodium chlorate, a chlorate salt, or a perchlorate salt, or a mixture thereof.

The method of the invention may further comprise producing carbon dioxide from the enclosed bioreactor and optionally processing the carbon dioxide in a useful manner, for example by providing the same as feedstock for a biomass-producing reactor as described in our co-pending U.S. Ser. No. 63,364,275, the contents of which are hereby incorporated by reference, for example.

The method may further comprise harvesting hydrogen from the enclosed bioreactor at a hydrogen harvesting rate, and separating the hydrogen from other gasses by filtering the hydrogen through a hydrogen-selective membrane filter and transferring the hydrogen into a hydrogen storage container.

Forming the synthetic reaction mixture may comprise adding the at least one hydrogen producing microorganism until a percentage of the hydrogen producing microorganism in the synthetic reaction mixture is at least about 10% or at least about 20% of a total amount of microorganisms in the synthetic reaction mixture.

The enclosed bioreactor may have a volume of at least about 100 m3, or at least about 103 m3, or at least about 104 m3, or at least about 105 m3. The enclosed bioreactor may have a volume of up to about 4×109 m3, or up to about 4×108 m3, or up to about 4×107 m3, or up to about 4×106 m3. The enclosed bioreactor may have a volume of from about 100 m3 to about 4×109 m3, or from about 100 m3 to about 4×106 m3, or from about 4×106 m3 to about 4×109 m3.

The hydrogen storage container may be a gas tank, a hydrogen subterranean formation, or a hydrogen artificial enclosure.

The hydrogen subterranean formation may include a natural formation or non-natural formation.

The hydrogen artificial enclosure may be made of one or more building materials. The building materials may include a cement, a plastic, a non-natural rubber, a geomembrane of any kind, concrete, a metal or metal alloy (such as steel), or a combination thereof.

The following examples are offered by way of illustration of certain embodiments of aspects of the application herein. None of the examples should be considered limiting on the scope of the application.

### EXAMPLES

### Example 1

Schematically illustrated (for a single well) in FIG. 1, two low producing oil wells are stimulated in a huff-n-puff application to increase microbial hydrogen production. A Berea sandstone core is conditioned under anaerobic conditions with an oil representative of an oil sample extractable from the well. Its indigenous microbiological content is determined, with the results set out in Tables 1 and 2 below:

TABLE 1
Sample 1—Indigenous microbial population:

| | |
|---|---|
| Halanaerobium praevalens DSM | 18.1% |
| 2228 | |
| Acinetobacter johnsonii | 13.4% |
| Desulfohalobium retbaense DSM | 12.1% |
| 5692 | |
| Halanaerobium hydrogeniformans | 7.4% |
| Methanohalophilus halophilus | 6.0% |
| Methanohalophilus mahii DSM | 4.7% |
| 5219 | |
| Escherichia coli | 2.0% |
| Halobacteroides halobius DSM | 2.0% |
| 5150 | |
| Azospirillum thiophilum | 1.3% |
| Keratinibaculum paraultunense | 1.3% |

TABLE 2
Sample 2—Indigenous microbial population:

| | |
|---|---|
| Methanohalophilus halophilus | 13.2% |
| Methanohalophilus mahii DSM | 11.0% |
| 5219 | |
| Halanaerobium praevalens DSM | 7.3% |
| 2228 | |
| Desulfohalobium retbaense DSM | 6.8% |
| 5692 | |
| Halanaerobium hydrogeniformans | 3.7% |
| Acinetobacter johnsonii | 3.2% |
| Petrotoga mobilis SJ95 | 3.2% |
| Halothermothrix orenii H 168 | 2.3% |
| Flexistipes sinusarabici DSM 4947 | 2.3% |
| Pelobacter acetylenicus | 1.8% |
| Methanotorris igneus Kol 5 | 1.4% |
| Bacillus mycoides | 1.4% |

In the first sample, nutrients are blended as described below in Table 3 into 500 bbls of produced water in a frac tank.

TABLE 3
Nutrient package mixed into the 500 bbls:

| Reagent | [g/L] |
|---|---|
| K₂HPO₄ | 1.044 |
| NH₄Cl | 1.5 |
| Sucrose | 1.41 |
| Yeast extract | 1.5 |
| Tween 80 | 0.081 |

The nutrient mix is injected down the annulus of the well (from which sample 1 is representative) and an additional 500 bbls of produced water is pumped down the annulus on top of the nutrient mixture. In the second well (of which sample 2 is representative), the same process occurs with the exception that a consortium of microbes capable of producing hydrogen from hydrocarbon fermentation, and the nutrient conditions capable of ensuring thriving of that consortia, is selected by means of core flood methodology and added to the first 500 bbls of produced water along with the nutrient package.

The consortium is prepared by combining hydrogen producing microorganisms selected by means of core flood methodology to be different from the indigenous microbial populations, and for their capability to digest hydrocarbons to yield hydrogen in preference to methane, in the proportions identified in Table 4:

TABLE 4
Well 2—Exogenous microbial population:

| | |
|---|---|
| Pseudothermotoga elfii | ~20% |
| Pseudothermotoga hypogea | ~20% |
| Thermotoga petrophila | ~20% |
| Petrotoga mobilis | ~20% |
| Caldanaerobacter tengcongensis | ~20% |

The exogenous microbes are maintained in anaerobic liquid culture and nurtured for 2 months under nitrogen (100% N2) at 150 F (65.56 deg C.), with fresh media inoculated every 3-4 days. The selected media is an ATCC 2114 medium modified for preferential culturing of extremophiles.

Approximately 400 L of microbial culture consisting of approximately 10⁸ cells/mL is added to the 500 bbls.

Following addition of the nutrient package (Well 1) and the nutrient/microbial consortium package (Well 2), the two wells are shut-in for 4 days. After the four-day shut-in period the wells are opened and samples are collected off the gas flow line for analysis with respect to H2 content on a gas chromatograph, with the results presented in Table 5 below:

TABLE 5
Gas Chromatography characterization of samples:

| | Baseline H2 | After shut-in |
|---|---|---|
| Well | (ppm) | H2 (ppm) |
| 1 (nutrients only) | <112 (LOD) | 1761 |
| 2 (nutrients and | <112 (LOD) | 13251 |
| microbes) | | |

The gas chromatography is carried out using a standard protocol as follows: 10 milliliter gas samples are extracted from culture bottles using 10 milliliter plastic luer lock syringes. Field gas samples are collected in multi-layer foil gas sampling bags connected via tygon tubing to a sampling valve directly off the of wellhead flow line. Gas samples are injected immediately into the inlet port of an SRI 8610C Gas Chromatograph. The sample is analyzed using a Flame Photometric Detector (FPD), a Flame Ionization Detector (FID), an FID with a large methanizer (FIDM), and a Thermal Conductivity Detector (TCD).

The samples are passed through an 18-inch HayeSep D Packed Column, a 3-foot Molecular Sieve 5A Packed Column, and then into the TCD and FIDM detectors following relay G injection. When relay F is turned on the samples are run through a 6-foot HayeSep D Column and a 60-meter MXT-1 Capillary Column before being analyzed using the FID and FPD. The G relay is turned on at time 0.020 minutes and is turned off at 1.000 minutes, while the F relay is turned on after 4.500 minutes. The initial temperature is set for 50° C. and held for 6 minutes before ramping to 270° C. at a rate of 30° C. per minute. The temperature is held at 270° C. for 6.500 minutes to remove excess sample from the columns.

Any peak areas produced are converted into ppm values using the trend lines of calibration curves derived from standards of various concentrations.

It will be seen from the results in Table 5 that modifying the composition of the well by the introduction into the well of a nutrient package and of consortium of hydrogen producing microorganisms selected by means of core flood methodology positively to diversify the microbiological abundance of hydrogen-producing microorganisms in the well and for the preferential production of hydrogen over methane increases hydrogen production from the well by two orders of magnitude with respect to baseline H2 production, and by an order of magnitude with respect to introduction of the nutrient package alone.

### Example 2: Microbe Laboratory Data

The consortium of microbes described in Example 1 and capable of producing hydrogen from hydrocarbon fermentation is used to inoculate 6 different synthetic seawater blends in triplicate as described below in Table 6.

TABLE 6
Synthetic seawater blends:

| Brine | Description |
|---|---|
| A | Synthetic seawater |
| B | Synthetic seawater with oil |
| C | Synthetic seawater with nutrients |
| D | Synthetic seawater with nutrients and oil |
| E | Synthetic seawater with enhanced nutrients |
| F | Synthetic seawater with enhanced nutrients and oil |
| G | Synthetic seawater with algae biomass and oil |

Synthetic seawater is a simple reproducible representative of produced water brines. It is produced using NeoMarine aquarium salts by Brightwell Aquatics. The oil used in this example is a sweet west Texas crude blend (API 25-35). 4 mL of the oil is used in 100 mL synthetic seawater sample. The nutrient packages employed are as follows in Tables 7, 8 and 9:

TABLE 7
Synthetic seawater with nutrients:

| Reagent | [g/L] | |
|---|---|---|
| Aquarium Salts | 35.40290621 | |
| K₂HPO₄ | 0.348 | |
| KH₂PO₄ | 0.227 | |
| NH₄Cl | 0.5 | |
| Wolfes Vitamin solution | 10 | mL |
| Reducing agent | 1 | |
| Resazurin solution | ~1 | mL |
| dH₂O | 989 | mL |

Combine, pH to desired 6.5 +− 0.5),
filter sterilize

TABLE 8
Synthetic seawater with enhanced nutrient package:

| Reagent | [g/L] | |
|---|---|---|
| Aquarium Salts | 35.40290621 | |
| K₂HPO₄ | 0.348 | |
| NH₄Cl | 0.5 | |
| Glucose | 0.47 | |
| Yeast extract | 0.5 | |
| Tween 80 | 0.027 | |
| Reducing agent | 1 | |
| Resazurin solution | ~1 | mL |
| dH₂O | 999 | mL |

Combine, pH to desired 6.5 +− 0.5),
filter sterilize

TABLE 9
Synthetic seawater with algae biomass nutrient package:

| Reagent | [g/L] | |
|---|---|---|
| Aquarium Salts | 35.40290621 | |
| K₂HPO₄ | 0 | |
| NH₄Cl | 0 | |
| Glucose | 0 | |
| Yeast extract | 0 | |
| Chlorella algae powder | 0.5 | |
| Tween 80 | 0.027 | |
| Reducing agent | 1 | |
| Resazurin solution | ~1 | mL |
| dH₂O | 999 | mL |

A 100 mL sample of each brine A-E is prepared anaerobically in glass bottles and sealed. Following inoculation, the bottles are incubated at 65 C for 48 hours along with abiotic controls for each brine.

At 48 hours, samples are taken for ATP analysis (microbial enumeration) and gas analysis, the results of which are shown in Table 10.

TABLE 10
ATP Analysis:

| | | Abiotic Control | | Inoculated | |
|---|---|---|---|---|---|
| | | H2 | Microbial | H2 | Microbial |
| | | Concen- | enumer- | Concen- | enumer- |
| | | tration | ation | tration | ation |
| Brine | Description | (ppm) | (cells/mL) | (ppm) | (cells/mL) |
| A | Synthetic seawater | 0 | 6.92E+03 | 0 | 1.51E+06 |
| B | Synthetic seawater | 0 | 2.00E+04 | 82.33 | 7.54E+06 |
| | with oil | | | | |
| C | Synthetic seawater | 0 | 1.40E+03 | 119 | 7.55E+06 |
| | with nutrients | | | | |
| D | Synthetic seawater | 0 | 7.67E+03 | 1406.7 | 2.58E+07 |
| | with nutrients and oil | | | | |
| E | Synthetic seawater | 0 | 9.80E+03 | 62.33 | 3.04E+07 |
| | with enhanced | | | | |
| | nutrients | | | | |
| F | Synthetic seawater | 0 | 2.95E+04 | 2735.33 | 2.07E+07 |
| | with enhanced | | | | |
| | nutrients and oil | | | | |
| G | Synthetic sea water | 0 | 1.18E+07 | 2365.5 | 1.01E+08 |
| | with algae biomass | | | | |
| | and oil | | | | |

In sample E, the enhanced nutrient package used causes rapid microbial growth at 24 hours and all of the carbon source is consumed which leads to a lower reading at 48 hours when no oil is present to maintain microbial activity, which rationalizes the lower H₂ concentration observed for this sample relative to comparative sample C.

The test kit used for the determination of ATP is the Luminultra QGO-M which is compliant with ASTM Standard E2694 for the measurement of ATP in Metalworking Fluids and D7687 for the measurement of ATP in fuels, fuel/water mixtures and fuel-associated water.

### Example 3: Core Flood Methodology

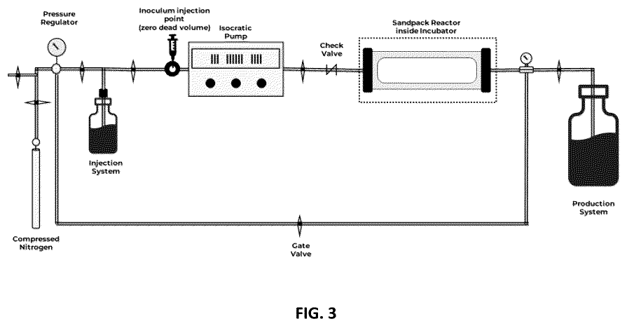

The microbial and nutrient package selected from Example 2 that can produce hydrogen from hydrocarbon fermentation is used to run dynamic and continuous injection studies at high pressure and temperature with core flood and sandpack equipment (FIG. 2 and FIG. 3).

Rock, sand, oil, and brine samples can be obtained from a potential well. The rock and sand sample can also be procured from outcrops or vendor sources that closely resemble the mineralogical and petrophysical properties of subsurface formations.

The components of these systems were procured from different suppliers. Some of the parts were customized to fit the requirements of the test.

All the components in the system are pressure tested for leakages. The system was ensured to be leakproof before starting an experiment.

The rock or sand sample is added to the reactor and closed on both sides. Vacuum pressure is applied to the reactor to remove any trapped air. Nitrogen gas is then injected into the reactor for a few hours to establish anaerobic conditions.

The pore pressure, confining pressure, and reservoir temperature are established using regulators and a convection oven. The representative rock core or sand is brought to representative fluid saturations by injecting anaerobic brine in the forward direction into the samples in the forward direction at high pressure and temperature. Live or dead crude oil is then injected into the rock or sand in the reverse direction at high pressure and temperature. If the rock or sand are already at representative fluid saturations, they will be used as is without modification.

The samples can be “aged” at reservoir pressure and temperature to replicate wettability conditions in the subsurface.

Brine is then injected into the rock or sand in the forward direction to establish fluid pressure. Effluent samples are run through a separator and then collected in separate evacuated collection chambers. The fluid samples can be analyzed discretely or continuously through in-line sensors or external measurement devices. Some of the parameters that would be analyzed include but not limited to microbial cell count, volatile fatty acids, effluent hydrocarbon composition, gas composition, gas generated volume, liquid volume, and surface adsorption.

Once the brine pressure is established, the reactors are inoculated by injecting microbes and nutrients into the brine stream in the forward direction at predetermined concentrations.

When sufficient inoculation is completed, the reactors may be shut-in for a period for microbial growth (otherwise known as a “Huff n' Puff” lab test). In continuous injection or a “waterflooding” lab test, there will be no shut-in.

After the shut-in period in the Huff n' Puff lab test, the flow lines are opened, and brine is injected into the reactor in the reverse direction. Fluid samples are continuously collected in separate evacuated collection chambers. The gas and liquids are then analyzed discretely or continuously.

In a waterflooding lab test, brine is continuously injected into the rock or sand. Microbes and nutrients can be added as a pill or continuously. Effluent fluid samples are continuously collected in separate evacuated collection chambers. They are then analyzed discretely or continuously.

The pressure drop across the reactor is continuously measured during the test.

The end of the test is predetermined based on injection volumes, injection time, soaking time, and data collection metrics.

Upon completing the test, the rock or sand samples are removed from the reactor and immediately stored in evacuated bottles for further DNA sequencing analysis. They are also analyzed for pore structure or sand surface analysis.

The reactors, flow lines, and collection chambers are then cleaned thoroughly with solvents and deionized water. They are made ready for the next round of testing.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

1. A process for the microbial production of hydrogen from the site of hydrocarbonaceous deposit, the process comprising:
modifying the composition of the deposit through the introduction into or into the vicinity of the deposit of at least one hydrogen producing microorganism,
wherein the at least one hydrogen producing microorganism or at least one microbiological condition conducive to the thriving of the microorganism is selected, or its selection is aided, by means of continuous flow methodology.

2. A process for according to claim 1, wherein the continuous flow methodology is core flood and/or sandpack methodology.

3. The process according to claim 1, wherein the core flood and/or sandpack methodology comprises
extracting a material sample from the site or selecting a material sample representative of a material from the site and subjecting the sample to analysis determinative of its response to microbiological conditions.

4. A process according to claim 3, wherein the material sample comprises a geological, mineralogical and/or a hydrocarbonaceous sample.

5. A process according to claim 4, wherein sample is extracted from the site of the hydrocarbonaceous deposit or otherwise selected to be representative—in at least one of its geochemical or geomineralogical characteristics—of on-site material.

6. The process according to claim 3, wherein the material sample is subjected to determinative analysis on or off the site of the hydrocarbonaceous deposit.

7. The process according to claim 3, wherein the determinative analysis comprises maintaining the sample under desirable conditions of temperature, pressure and/or chemical environment and determining a response of the sample to one or more microbiological conditions.

8. The process according to claim 7, wherein the desirable conditions at least to some extent replicate one or more of the those conditions (of temperature, pressure and/or chemical environment) of the site location from which the sample is extracted.

9. The process according to claim 7, The process of claim 7, where in the desirable conditions comprise a pressure of up to about 2,000 psi, up to about 4,000 psi, up to about 6,000 psi, up to about 8,000 psi, or up to about 10,000 psi.

10. The process according to claim 3, wherein the sample extraction or sample selection is conducted under anaerobic conditions.

11. The process according to claim 10, wherein the extracted sample is maintained under anaerobic conditions in any interim period between sample extraction or selection and its subjection to core flood and/or sandpack methodology.

12. The process according to claim 1, wherein the core flood and/or sandpack methodology takes place under anaerobic conditions.

13. The process according to claim 1, wherein the hydrogen producing microorganism is selected by means of core flood and/or sandpack methodology positively to diversify the microbial abundance of hydrogen-producing microorganisms in the deposit when charged thereto.

14. The process according to claim 1, wherein the hydrogen producing microorganism is selected by means of core flood and/or sandpack methodology for the preferential production of hydrogen over methane.

15. The process according to claim 1, wherein the at least one hydrogen producing microorganism is:
a. a microorganism not naturally present in the hydrocarbonaceous deposit; and/or
b. of a strain of microorganisms not naturally present in the hydrocarbonaceous deposit; and/or
c. of a species of microorganisms not naturally present in the hydrocarbonaceous deposit; and/or
d. of a genus of microorganisms not naturally present in the hydrocarbonaceous deposit;
e. a microorganism naturally present in the hydrocarbonaceous deposit; and/or
f. a microorganism naturally present in the hydrocarbonaceous deposit but genetically modified to increase (relative to the naturally present microorganism) its propensity for hydrogen production by the metabolization by that microorganism of one or more hydrocarbons contained within the deposit.

16. The process according to claim 1, wherein the at least one hydrogen producing microorganism is one of a plurality of different hydrogen producing microorganisms, strains of microorganisms, species of microorganisms, genera of microorganisms and/or naturally occurring, optionally genetically modified, organisms introduced into the deposit.

17. A process according to claim 16, wherein the plurality is greater than two, greater than three, greater than four, greater than five and/or greater than ten.

18. The process according to claim 1, wherein the at least one hydrogen producing microorganism is introduced into the deposit and accompanied during, after or upon its introduction by at least one nutrient selected to promote the growth of said microorganism and introduced into the deposit for that purpose, optionally wherein the at least one nutrient is are supplied from a nutrient reservoir

19. A process according to claim 18, wherein characteristics of the at least one nutrient are determined at least in part by core flood and/or sandpack methodology.

20. The process according to claim 1, wherein the at least one hydrogen producing microorganism is introduced into the deposit and accompanied during, after or upon its introduction by at least one pH regulator selected to regulate the pH environment in which the microorganism resides in the deposit and introduced into the deposit for that purpose.

21-31. (canceled)
